(19) United States
(12) Patent Application Publication
(10) Pub. No.: US 2025/0057684 A1
(43) Pub. Date: Feb. 20, 2025

(54) ABSORBENT PENILE ENCLOSURE

(71) Applicant: Bebi Shelliza Ballee Goolsarran, Miramar, FL (US)

(72) Inventor: Bebi Shelliza Ballee Goolsarran, Miramar, FL (US)

(21) Appl. No.: 18/235,648

(22) Filed: Aug. 18, 2023

(51) Int. Cl. A61F 5/453; A61F 5/44

(52) U.S. Cl. CPC A61F 5/453; A61F 5/4401; A61F 5/4404

(57) ABSTRACT

An absorbent penile enclosure that includes an inner layer defining an entrance aperture and of a permeable material fabric material enclosing a cavity spatially coupled to the enclosed entrance aperture and having an inner layer thickness, an intermediate layer superimposed over and enclosing the inner layer, having an intermediate layer thickness greater than the inner layer thickness, and of a fabric absorbent material, and an exterior layer superimposed over and enclosing the intermediate layer, having an exterior layer thickness less than the intermediate layer thickness, of an impervious material, the exterior layer defining two terminal opposing ends of the absorbent enclosure and defining an enclosure length separating the two terminal opposing ends that spans approximately 38-203 mm.

### FIELD OF THE INVENTION

The present invention relates generally to an absorbent article for enclosing a male's genitalia and, more particularly, relates to selectively couplable enclosure or pad for a male's penis that is configured to absorb urine or other bodily fluids emitted therefrom.

### BACKGROUND OF THE INVENTION

Incontinence is a very common issue prevalent among many male individuals. Even if a male user is not incontinent, inadvertent urine leakage is an issue many male users experience. These issues are extremely problematic due to the adverse effects on an individual's clothing, in addition to the emotional effects they cause to the individual, personally. Some known penile enclosures directed toward absorbing urine include forming a cylindrical enclosure with an absorbent material, e.g., cotton, with a waterproof layer surrounding said absorbent material. These penile enclosures, while effective for absorbing urine, problematically leave a significant amount of moisture in direct contact with the user's skin. This moisture also increases the likelihood of the enclosure being inadvertently removed. Notwithstanding the moisture in direct contact with the user's skin, these penile enclosures also become routinely and inadvertently removed simply because of their structural configuration and attachment to the user.

To that end, some known penile enclosures have been created to more securely fasten to the user, including using elastic bands, hook-and-loop couplers, or adhesive. Each of these known enclosures are problematic in that they only temporarily secure the enclosure to the user, do not adequately secure the enclosure to the user when the user is more active, and/or over-designed causing them to be comparatively costly, difficult for consumers to figure out how to utilize, and time consuming to attach and detach from the user.

Therefore, a need exists to overcome the problems with the prior art as discussed above.

### SUMMARY OF THE INVENTION

The invention provides an absorbent penile enclosure that overcomes the hereinafore-mentioned disadvantages of the heretofore-known devices and methods of this general type and that effectively absorbs and retains urine in the enclosure for disposal. Furthermore, the present invention ensures a secure attachment of the enclosure to the user, even while more active.

With the foregoing and other objects in view, there is provided, in accordance with the invention, an absorbent penile enclosure is disclosed that includes an inner layer defining an entrance aperture and of a permeable material fabric material enclosing a cavity spatially coupled to the enclosed entrance aperture and having an inner layer thickness, an intermediate layer superimposed over and enclosing the inner layer, having an intermediate layer thickness greater than the inner layer thickness, and of a fabric absorbent material, and an exterior layer superimposed over and enclosing the intermediate layer, having an exterior layer thickness less than the intermediate layer thickness, of an impervious material, wherein the exterior layer defines two terminal opposing ends of the absorbent enclosure and defines an enclosure length separating the two terminal opposing ends that spans approximately 38-203 mm.

In accordance with a further feature of the present invention, the inner layer is infused with an aloe material, is of a polyester fiber material, and is of a moisture wicking material.

In accordance with yet another feature of the present invention, the exterior layer is of a polyethylene material.

In accordance with a further feature of the present invention, the inner layer, the intermediate layer, and the exterior layer continuously span the enclosure length.

In accordance with an exemplary feature of the present invention, the inner layer is approximately 0.5-1.5 mm in thickness, the intermediate layer is approximately 1-2.5 mm in thickness, and the exterior layer is approximately 0.1-0.4 mm in thickness.

In accordance with an additional feature of the present invention, the inner layer and the intermediate layer are of a thickness less than approximately 3 mm.

In accordance with another feature, an embodiment of the present invention includes an adhesive attachment film strip disposed on an outer surface of the exterior layer and substantially spanning the enclosure length.

In accordance with yet another feature, an embodiment of the present invention also includes a release liner superimposed over and coupled to the adhesive film strip, operably configured to be removed from and expose the adhesive film strip, and longitudinally spanning substantially the enclosure length.

In accordance with a further feature, an embodiment of the present invention also includes the release liner having a removal tab portion uncoupled to the adhesive film strip, operably configured to be removed from and expose the adhesive film strip, and longitudinally spanning substantially the enclosure length.

In accordance with an additional feature, an embodiment of the present invention also includes a removable attachment strip of a flexible material, with a first end coupled to an outer surface of the exterior layer, a second free end opposing the first end of the removable attachment strip, a removable strip length separating the first end and second free end, an outer surface, an inner surface opposing the outer surface of the removable attachment strip, having an adhesive material disposed on the inner surface of the removable attachment strip, and operably configured to at least partially surround and adhesively couple to a circumference of the outer surface of the exterior layer.

In accordance with a further feature, an embodiment of the present invention also includes a tube defining an entrance aperture disposed within the cavity, proximal to one of the two terminal opposing ends of the absorbent enclosure, and proximal to the inner layer, defining an exit aperture, and a tube channel separating the entrance and exit apertures of the tube, wherein the tube is operably configured to transport liquid from the entrance aperture of the tube through the tube channel and out through the exit aperture of the tube.

The present invention may also be described as including an absorbent penile enclosure with a unitary body with a first terminal end defining an enclosed entrance aperture, a distal terminal end opposing the first terminal end, an enclosure length separating the first and distal terminal ends, with an inner layer of a permeable moisture wicking material fabric material enclosing a cavity, with an intermediate layer superimposed over and enclosing the inner layer and of a fabric absorbent material, and with an exterior layer superimposed over and enclosing the intermediate layer and of an impervious material, wherein the enclosed entrance aperture is shaped and sized to provide a single penile pathway into the cavity and the inner layer, intermediate layer, and exterior layer spanning the enclosure length.

In accordance with an additional feature of the present invention, the unitary body is formed into a cylindrical shape.

Although the invention is illustrated and described herein as embodied in an absorbent penile disclosure, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic of the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time. Also, for purposes of description herein, the terms “upper”, “lower”, “left,” “rear,” “right,” “front,” “vertical,” “horizontal,” and derivatives thereof relate to the invention as oriented in the figures and is not to be construed as limiting any feature to be a particular orientation, as said orientation may be changed based on the user's perspective of the enclosure. Furthermore, there is no intention to be bound by any expressed or implied theory presented in the preceding technical field, background, brief summary or the following detailed description.

As used herein, the terms “about” or “approximately” apply to all numeric values, whether or not explicitly indicated. These terms generally refer to a range of numbers that one skill in the art would consider equivalent to the recited values (i.e., having the same function or result). In many instances these terms may include numbers that are rounded to the nearest significant figure. Unless otherwise stated herein, approximately may at least be interpreted as +/−5 mm if expressed in mm and +/−2% if expressed in percentage. In this document, the term “longitudinal” should be understood to mean in a direction corresponding to an elongated direction of the enclosure, or in direction spanning from the opening of the enclosure to the distal tip of the enclosure, wherein “traverse” should be understood to mean in a side-to-side direction or 180°+/−100 relative to the longitudinal direction.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a perspective view of an absorbent penile enclosure in accordance with one embodiment of the present invention;

FIG. 2 is an elevational front view of the absorbent penile enclosure in FIG. 1 disposed within its packaging in accordance with one embodiment of the present invention;

FIG. 3 is an elevational front view of the absorbent penile enclosure in FIG. 1;

FIG. 4 is an elevational side view of the absorbent penile enclosure in FIG. 1;

FIG. 5 is a cross-sectional view of the absorbent penile enclosure in FIG. 4 and along section line 5-5;

FIG. 6 is a perspective view of an absorbent penile enclosure with a catheter-like tube in accordance with one embodiment of the present invention;

FIG. 7 is a cross-sectional view of the absorbent penile enclosure in FIG. 6;

FIG. 8 is a perspective view of an absorbent penile enclosure in accordance with one embodiment of the present invention;

FIG. 9 is an elevational front view of the absorbent penile enclosure in FIG. 8 disposed within its packaging in accordance with one embodiment of the present invention;

FIG. 10 is an elevational front view of the absorbent penile enclosure in FIG. 8 and with the removable attachment strip in a removed and open position in accordance with one embodiment of the present invention;

FIG. 11 is a cross-sectional view of the absorbent penile enclosure in FIG. 10 and along section line 11-11; and

FIG. 12 is a perspective view of an absorbent penile enclosure with a catheter-like tube in accordance with one embodiment of the present invention.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

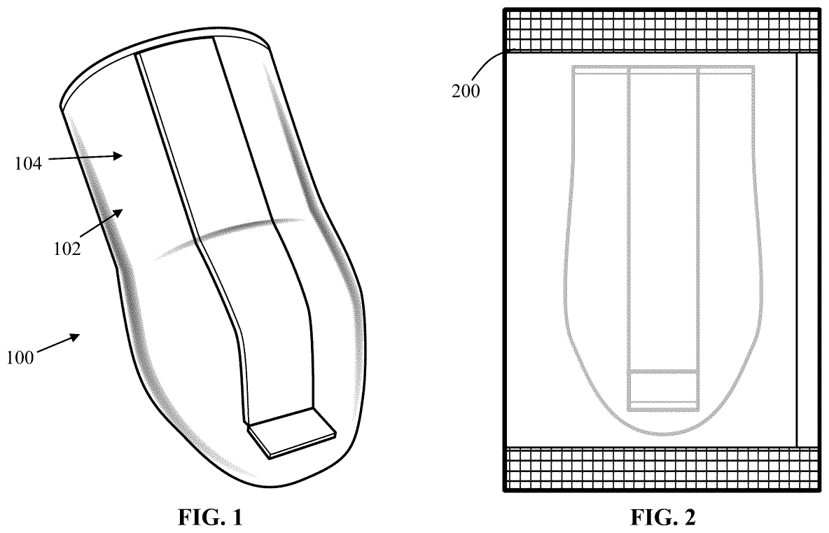

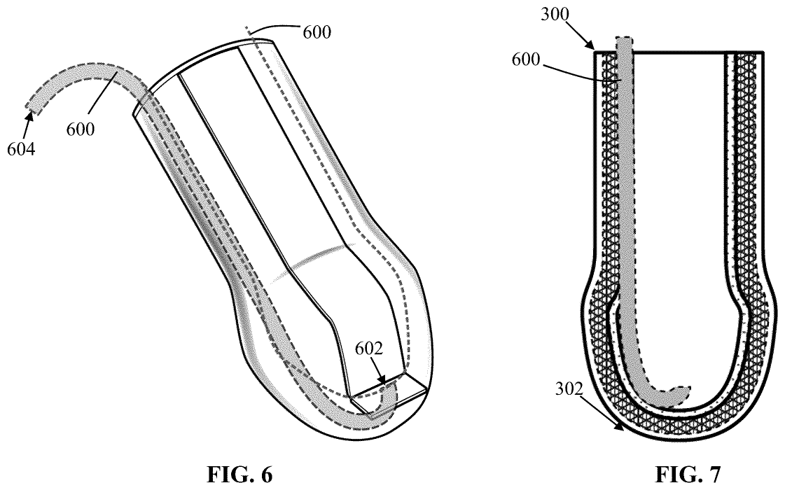

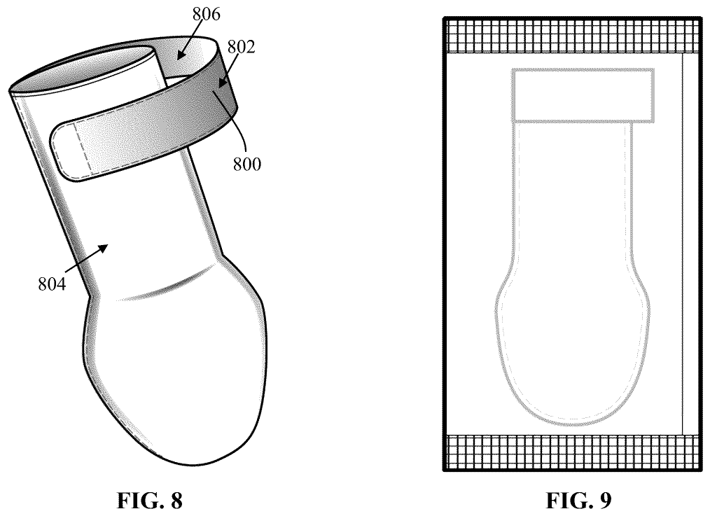

The present invention provides a novel and efficient absorbent penile enclosure configured to effectively and securely absorb urine and other liquids from a user's penis. Referring now to FIGS. 1-5, one exemplary embodiment of the present invention is shown in various views and designed for a child. FIGS. 6-7 depict an exemplary embodiment of the present invention designed for an adolescent or adult. FIGS. 8-12 depict other exemplary embodiments for an adolescent or adult. Regardless the target user, the above-referenced figures show several advantageous features of the present invention, but, as will be described below, the invention can be provided in several shapes, sizes, combinations of features and components, and varying numbers and functions of the components. With reference to FIGS. 1-5 by way example, an absorbent penile enclosure 100 is disclosed. The enclosure 100 may include a body 102 that is formed with multiple layers 500, 502, 504 that are unitary in nature, i.e., operating and functioning as a single or uniform unit, which also includes the enclosure moving and being applied as a single structure. The body 102 is also preferably formed into a cylindrical shape with a bulbous distal end portion (as best seen in FIG. 7) to ensure a more snug fit with the user.

The body 102 may include two opposing and terminal ends 300, 302 that define an enclosure length 304. The enclosure length 304 preferably spans approximately 38-203 mm, or 1.5-6 in so as to provide an adequate and desired length for the user (wherein, beneficially, complete coverage of the user's penis is not required to receive the functional benefit of the enclosure 100). If the enclosure length 304 is too long, it may cause the enclosure to be inadvertently dislodged. Preferably, the enclosure length is approximately 44 mm (+/−5 mm), or 1.8 in (+/−1.6-2.0 in) for child and 91 mm (+/−5 mm), or 3.6 in (+/−0.2 in) for an adolescent or adult. In one embodiment, all three layers 500, 502, 504 span the entire enclosure length 304. In other embodiments, just one of the layers 500, 502, 504 span the enclosure length 304.

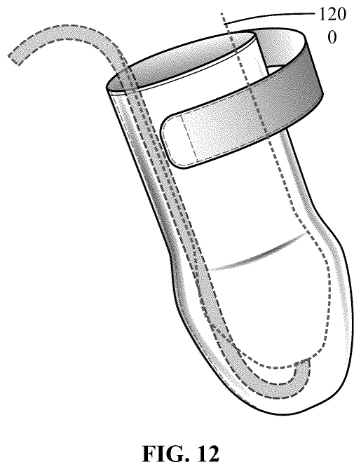

With reference specifically to FIG. 5, the enclosure 100 beneficially includes three layers, an inner layer 500, an intermediate layer 502 superimposed over the inner layer 500, and an exterior layer 504 superimposed over the intermediate layer 502. Each layer may be directly coupled to each other using adhesive or heat. In one embodiment, the inner layer 500 defines an entrance aperture 506 and defines and encloses a cavity 508 that is spatially coupled to the entrance aperture 506, i.e., the user's penis (exemplified with outlines 600, 1200 in FIG. 6 and FIG. 12) is inserted into and through the entrance aperture 506 and disposed into the cavity 508 until reaching or being disposed proximal to an internal distal surface 510. The term “proximal” is defined as located at or near, within 25 mm from, the referencing surface or structure. Said differently, the enclosed entrance aperture 506 is beneficially shaped and sized (e.g., a circular shape having a diameter of approximately 25-100 mm) to provide a single penile pathway into the cavity 508.

The inner layer 500 is beneficially of a permeable material fabric material, wherein said material enclosing the cavity 508. The inner layer 500 is moisture wicking, in that it is configured to transport liquid therethrough. Some exemplary, yet effective, materials for the inner layer 500 include merino wool, bamboo, nylon, or polyester fiber. As the inner layer 500 will directly contact the user's penis, it may beneficially be infused with aloe material. The inner layer 500 has a thickness that is generally less than the intermediate layer 502 and greater than the exterior layer 504.

The intermediate layer 502 is superimposed over and also encloses the inner layer 500, preferably through the entire enclosure length 304 where the inner layer 500 is located. The intermediate layer 502 has an intermediate layer thickness that is greater than the inner layer thickness, as the intermediate layer 502 is designed and configured to retain any emitted liquid. Beneficially, the intermediate layer 502 is made of a fabric absorbent material to retain said liquid and is more absorbent and not as moisture wicking as the inner layer 500.

The exterior layer 504 is also superimposed over and encloses the intermediate layer 502. The exterior layer 504 has an exterior layer thickness that is less than the intermediate layer thickness and less than the thickness of the inner layer 500. The exterior layer 504 is beneficially of an impervious material, thereby preventing the retained liquid in the intermediate layer 502 from escaping the enclosure 100 through the outermost layer. In one embodiment, the exterior layer 504 defines the two terminal opposing ends 300, 302 of the enclosure 100 and principally defines the enclosure length 304. separating the two terminal opposing ends 300, 302 that spans approximately 38-203 mm (1.5-6 in). In one embodiment, the exterior layer 504 is made of a polyethylene material, e.g., a polyethylene film.

To ensure a lightweight, yet effective, enclosure 100, the inner layer 500 is approximately 0.5-1.5 mm (0.02-0.06 in) in thickness, the intermediate layer 502 is approximately 1-2.5 mm (0.04-0.1 in) in thickness, and the exterior layer 504 is approximately 0.1-0.4 mm (0.004-0.02 in) (preferred is 0.25 mm) in thickness. In preferred embodiments, the thickness of each layer is the same spanning the enclosure length 304. Furthermore, the inner layer 500 and the intermediate layer 502 are also preferably of a thickness less than approximately 3 mm (0.11 in). With reference to FIG. 2, the enclosure 100 is preferably stored in a watertight and/or hermetically sealed package 200 that is configured to be selectively opened by the user before use, thereby preserving the integrity of the adhesive and other contents of the enclosure 100.

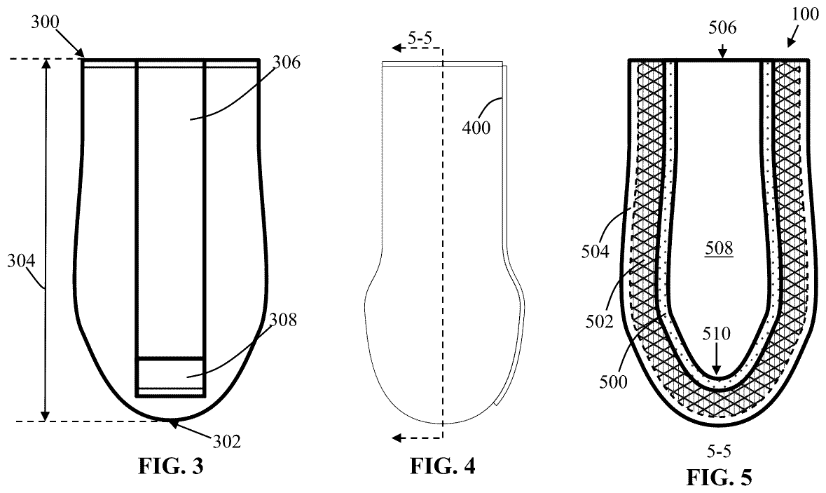

As seen best in FIG. 1, FIG. 3, and FIGS. 6-7, an adhesive attachment film strip 400 is disposed on an outer surface 104 of the exterior layer 504 and substantially spans, i.e., 75% or greater, the enclosure length 304. In other embodiments, the adhesive attachment film strip 400 is located on the inner layer 500 and couplable to a user's penis. When on the outer surface 104, the adhesive attachment film strip 400 beneficially enables the enclosure 100 to adhesively couple to the user's clothing, without damaging the user's clothes and securing the enclosure when the user is more active. To that end, the adhesive attachment film strip 400 is made of a silicone-based or polymer-based material. The adhesive attachment film strip 400 is slender and continuously and substantially spans the enclosure length 304. For example, the adhesive attachment film strip 400 may span 45 mm and be 5-12 mm in width that may be uniform.

The adhesive attachment film strip 400 beneficially includes a release liner 306 superimposed over and coupled to the adhesive film strip 400, is operably configured to be removed from and expose the adhesive film strip 400, and also longitudinally spans substantially the enclosure length 304. The release liner 306 is preferably of a paper material conducive to being removed without removing the adhesive. The release liner 306 may include a removal tab portion 308 uncoupled to the adhesive film strip 400, is operably configured to be removed from and expose the adhesive film strip 400, and that longitudinally spans substantially (75% or greater) the enclosure length 304. The release liner 306 is completely removable from the exterior layer 504 to expose the adhesive strip 400.

As seen best in FIGS. 8-12, the enclosure 100 may also include a removable attachment strip 800 of a flexible material, similar to the material for the exterior layer 504. The attachment strip 800 may be completely removable or, preferably, partially removable relative to the exterior layer 504. The attachment strip 800 includes a first end 1000 coupled (preferably permanently coupled) to an outer surface 804 of the exterior layer 504, a second free end 1002 opposing the first end 1000 of the removable attachment strip 600, and a removable strip length (approximately 25-50 mm) separating the first end 1000 and second free end 1002. The second end 802 is “free” in that it is not permanently coupled to any external structure or surface, and can be selectively removed from the exterior layer 504. The removable attachment strip 800 includes an outer surface 802, an inner surface 806 opposing the outer surface 802 of the removable attachment strip 800, includes an adhesive material (like the adhesive used for the attachment film strip 400) disposed on the inner surface 806 of the removable attachment strip 800, and is operably configured to at least partially surround and adhesively couple to a circumference of the outer surface 804 of the exterior layer 504.

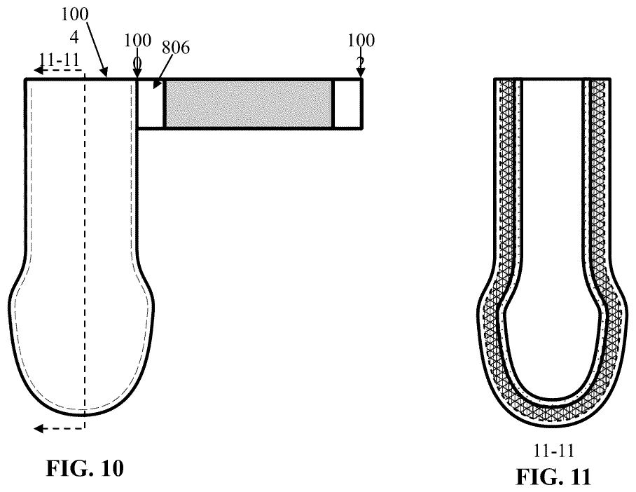

The removable attachment strip 800 also may include adhesive of a silicone-based or polymer-based material and is preferably traversely oriented relatively to the longitudinal orientation of the enclosure (as best seen in FIG. 10). The attachment strip 800 has dimensions akin to the adhesive attachment film strip 400 and beneficially provides a compressive force on the shaft of a user's penis, securing the enclosure thereto. The attachment strip 800 is preferably proximal and traversely oriented to the terminal end 1004 of the enclosure. The outer surface 804 may also include an adhesive material disposed thereon for securing the enclosure to a user's clothes. The adhesive on the attachment strip 800 may be completely disposed thereon, e.g., over the entire surface area, or partially disposed thereon, preferably on a central portion of the surface of the attachment strip 800 (as best seen in FIG. 10).

In one embodiment of the present invention and as best seen in FIGS. 5-7, the enclosure 100 includes a tube 600 defining an entrance aperture 602, defining an exit aperture 604, and a tube channel separating the entrance and exit apertures 602, 604 of the tube 600. The entrance aperture 602 is preferably disposed within the cavity 508, proximal to one of the two terminal opposing ends 300, 302 of the absorbent enclosure 100, and proximal to the inner layer 500 (as exemplified best in FIG. 7). The tube 600 is operably configured to transport liquid from the entrance aperture 602 of the tube 600 through the tube channel and out through the exit aperture 604 of the tube 600. The discharge into the tube 600 may lead to a drainage bag that is selectively drained by the user.

As such, an absorbent penile enclosure is disclosed that safely, effectively, and securely enables liquid discharge to be removed and prevents damage or moisture from reaching the user's clothes. The enclosure includes an adhesive on a side of the enclosure for attachment to a user's clothing or to the user.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the above-described features.

What is claimed is:

1. An absorbent penile enclosure comprising:
an inner layer defining an entrance aperture and of a permeable material fabric material enclosing a cavity spatially coupled to the enclosed entrance aperture and having an inner layer thickness;
an intermediate layer superimposed over and enclosing the inner layer, having an intermediate layer thickness greater than the inner layer thickness, and of a fabric absorbent material; and
an exterior layer superimposed over and enclosing the intermediate layer, having an exterior layer thickness less than the intermediate layer thickness, of an impervious material, the exterior layer defining two terminal opposing ends of the absorbent enclosure and defining an enclosure length separating the two terminal opposing ends that spans approximately 38-203 mm.

2. The absorbent penile enclosure according to claim 1, wherein the inner layer is infused with an aloe material.

3. The absorbent penile enclosure according to claim 1, wherein the inner layer of a polyester fiber material.

4. The absorbent penile enclosure according to claim 1, wherein the exterior layer is of a polyethylene material.

5. The absorbent penile enclosure according to claim 1, wherein the inner layer is of a moisture wicking material.

6. The absorbent penile enclosure according to claim 1, wherein the inner layer, the intermediate layer, and the exterior layer continuously span the enclosure length.

7. The absorbent penile enclosure according to claim 1, wherein the inner layer is approximately 0.5-1.5 mm in thickness, the intermediate layer is approximately 1-2.5 mm in thickness, and the exterior layer is approximately 0.1-0.4 mm in thickness.

8. The absorbent penile enclosure according to claim 7, wherein the inner layer and the intermediate layer are of a thickness less than approximately 3 mm.

9. The absorbent penile enclosure according to claim 1, further comprising:
an adhesive attachment film strip disposed on an outer surface of the exterior layer and substantially spanning the enclosure length.

10. The absorbent penile enclosure according to claim 9, further comprising:
a release liner superimposed over and coupled to the adhesive film strip, operably configured to be removed from and expose the adhesive film strip, and longitudinally spanning substantially the enclosure length.

11. The absorbent penile enclosure according to claim 10, wherein the release liner further comprises:
a removal tab portion uncoupled to the adhesive film strip, operably configured to be removed from and expose the adhesive film strip, and longitudinally spanning substantially the enclosure length.

12. The absorbent penile enclosure according to claim 1, further comprising:
a removable attachment strip of a flexible material, with a first end coupled to an outer surface of the exterior layer, a second free end opposing the first end of the removable attachment strip, a removable strip length separating the first end and second free end, an outer surface, an inner surface opposing the outer surface of the removable attachment strip, having an adhesive material disposed on the inner surface of the removable attachment strip, and operably configured to at least partially surround and adhesively couple to a circumference of the outer surface of the exterior layer.

13. The absorbent penile enclosure according to claim 1, further comprising:
a tube defining an entrance aperture disposed within the cavity, proximal to one of the two terminal opposing ends of the absorbent enclosure, and proximal to the inner layer, defining an exit aperture, and a tube channel separating the entrance and exit apertures of the tube, the tube operably configured to transport liquid from the entrance aperture of the tube through the tube channel and out through the exit aperture of the tube.

14. An absorbent penile enclosure comprising:
a unitary body with a first terminal end defining an enclosed entrance aperture, a distal terminal end opposing the first terminal end, an enclosure length separating the first and distal terminal ends, with an inner layer of a permeable moisture wicking material fabric material enclosing a cavity, with an intermediate layer superimposed over and enclosing the inner layer and of a fabric absorbent material, and with an exterior layer superimposed over and enclosing the intermediate layer and of an impervious material, wherein the enclosed entrance aperture is shaped and sized to provide a single penile pathway into the cavity and the inner layer, intermediate layer, and exterior layer spanning the enclosure length.

15. The absorbent penile enclosure according to claim 14, further comprising:
an inner layer thickness of the inner layer;
an intermediate layer thickness of the intermediate layer greater than the inner layer thickness; and
an exterior layer thickness of the exterior layer less than the intermediate layer thickness and the inner layer thickness, combined.

16. The absorbent penile enclosure according to claim 14, wherein the unitary body is formed into a cylindrical shape.

17. The absorbent penile enclosure according to claim 14, wherein the enclosure length spans approximately 38-203 mm.

18. The absorbent penile enclosure according to claim 14, further comprising:
an adhesive attachment film strip disposed on an outer surface of the exterior layer and substantially spanning the enclosure length.

19. The absorbent penile enclosure according to claim 14, further comprising:
a removable attachment strip of a flexible material, with a first end coupled to an outer surface of the exterior layer, a second free end opposing the first end of the removable attachment strip, a removable strip length separating the first end and second free end, an outer surface, an inner surface opposing the outer surface of the removable attachment strip, having an adhesive material disposed on the inner surface of the removable attachment strip, and operably configured to at least partially surround and adhesively couple to a circumference of the outer surface of the exterior layer.
